(19) United States
(12) Patent Application Publication
(10) Pub. No.: US 2019/0346575 A1
(43) Pub. Date: Nov. 14, 2019

(54) LIGHT GUIDING IN AN X-RAY DETECTOR

(71) Applicant: KONINKLIJKE PHILIPS N.V., EINDHOVEN (NL)

(72) Inventor: THOMAS KOEHLER, NORDERSTEDT (DE)

(21) Appl. No.: 16/466,307

(22) PCT Filed: Nov. 28, 2017
(86) PCT No.: PCT/EP2017/080620

§ 371 Date: Jun. 4, 2019

(30) Foreign Application Priority Data
Dec. 8, 2016 (EP) 16202950.8

(51) Int. Cl. G01T 1/20; A61B 6/00

(52) U.S. Cl. CPC G01T 1/2002; A61B 6/4291; G01T 1/2018; A61B 6/4208

(57) ABSTRACT

The present invention relates to light guiding in an X-ray detector. In order to provide a focussing of a scintillator, an X-ray detector (30) is provided comprising a scintillator arrangement (32) in a first layer (34), an optical guide arrangement (36) in a second layer (38), and a light-sensitive sensor arrangement (40) in a third layer (42). The second layer is provided between the first and the third layer. The scintillator arrangement comprises a structured scintillator with a plurality of scintillator elements (44) arranged as trenches separated from each other by wall elements (46) in a grating structure; wherein the trenches are configured to receive X-ray radiation from one side and wherein the trenches are configured to emit visible light on a second side. The trenches are arranged focused on a common point in which an X-ray source is arrangeable. The light-sensitive sensor arrangement comprises a plurality of sensor elements (48) that detect light generated by the scintillator elements. The optical guide arrangement comprises a light guiding plate (10), the light guiding plate comprising a plurality of light guide elements (12) arranged extending between a first surface (14) of the light guiding plate and an opposite second surface (16) of the light guiding plate. The first surface is configured for entry (18) of light generated by the scintillator to be arranged on top of the light guiding plate; and wherein the second surface is configured for exit (20) of the light towards the light-sensitive sensor arrangement being arranged adjacent the light guiding plate. the light guide elements are configured to guide light from the first surface to the second surface. Further, the first surface and the second surface are provided at least partly inclined relative to each other.

### FIELD OF THE INVENTION

The present invention relates to a light guiding plate for an X-ray detector, to an X-ray detector, to an X-ray imaging system and to a method for detecting X-ray radiation for medical X-ray imaging.

### BACKGROUND OF THE INVENTION

In X-ray imaging, phase-contrast and dark-field imaging is of increasing interest, for example, for a 2D radiography examination of the lung. Since a rather large absorber grating would be needed, structured scintillators have been developed where the scintillator itself is provided as a grating that converts incident X-ray radiation mainly from a certain range of direction only. For example, “A low cost method for hard x-ray grating interferometry” (Yang Du et al., November 2016; IPO publishing, Institute of Physics and Engineering in Medicine; Phys. Med. Biol. 61 (2016) pages 8266-8275) describes a structured scintillator. However, a high aspect ratio of the scintillator portions, which is needed for providing a sufficient signal in dark field X-ray imaging, reduces the range of incoming photons, i.e. the range of incoming and effective radiation.

### SUMMARY OF THE INVENTION

There may thus be a need to provide a focussing of the scintillator.

The object of the present invention is solved by the subject-matter of the independent claims; further embodiments are incorporated in the dependent claims. It should be noted that the following described aspects of the invention apply also for the light guiding plate for an X-ray detector, for the X-ray detector, for the X-ray imaging system and for the method for detecting X-ray radiation for medical X-ray imaging.

According to the present invention, an X-ray detector is provided. The X-ray detector comprises a scintillator arrangement in a first layer, an optical guide arrangement in a second layer and a light-sensitive sensor arrangement in a third layer. The second layer is provided between the first and the third layer. The scintillator arrangement comprises a structured scintillator with a plurality of scintillator elements arranged as trenches separated from each other by wall elements in a grating structure; wherein the trenches are configured to receive X-ray radiation from one side and wherein the trenches are configured to emit visible light on a second side. The light-sensitive sensor arrangement comprises a plurality of sensor elements that detect light generated by the scintillator elements. Further, the optical guide arrangement comprises a light guiding plate for an X-ray detector device. The light guiding plate comprises a plurality of light guide elements arranged extending between a first surface of the light guiding plate and an opposite second surface of the light guiding plate. The first surface is configured for entry of light generated by the scintillator to be arranged on top of the light guiding plate; and wherein the second surface is configured for exit of the light towards the light sensitive sensor arrangement being arranged adjacent the light guiding plate. The light guides elements are configured to guide light from the first surface layer to the second surface layer. Further, the first surface and the second surface are provided at least partly inclined relative to each other. The trenches are arranged focussed on a common point in which an X-ray source is arrangeable. Tthe light-sensitive sensor arrangement comprises a plurality of sensor elements that detect light generated by the scintillator elements.

By arranging the two surfaces inclined to each other, a focussing of a scintillator to a desired range of direction for incoming photons is provided. This is in particular suitable for structured scintillators that have an implicit direction in their sensitivity.

According to an example, the second surface is arranged as a plane surface and the first surface is configured as a concave surface.

The concave arrangement can be a continuous concave shape or can be a polygonal shape with sections of plane parts.

According to an example, the first surface is arranged to be aligned to a focus.

The light guiding structure can thus be focussed on an X-ray source for example.

According to an example, a plurality of plate segments is provided, which are provided with different inclination of their first surface in relation to their second surface.

Hence, a facilitated manufacture and assembly is provided.

According to an example, the light guide elements are provided as optical fibres configured to guide and transmit light between their ends. The optical fibres provide total reflection along their outer boundaries for light rays guided from the first to the second surface within the fibre.

According to an example, the light guiding plate comprises a light transparent base material arranged within a grid-like pattern of wall segments that provide total reflection for light rays guided from the first to the second surface.

According to the present invention, also an X-ray imaging system is provided. The X-ray imaging system comprises an X-ray source and an X-ray detector. The X-ray source is configured to generate X-ray radiation that is detectable by the X-ray detector. The X-ray detector is provided as an X-ray detector according to one of the examples above.

In an example, the X-ray imaging system is a dark-field X-ray imaging system which is configured for photon counting based on coherent small scattering of X-rays due to phase stepping.

According to the present invention, also a method for detecting X-ray radiation for medical X-ray imaging is provided. The method comprises the following steps: a) receiving X-ray radiation on a first side in a scintillator arrangement in a first layer having a structured scintillator with a plurality of scintillator elements arranged as trenches separated from each other by wall elements in a grating structure; wherein the trenches are arranged focused on a common point in which an X-ray source is arrangeable; wherein the X-ray radiation is at least partly transferred by generating visible light;

b) emitting the visible light by the scintillator arrangement on a second side;

c) receiving and guiding the visible light from a first surface of an optical guide arrangement configured for entry of light generated by the scintillator to a second surface configured for exit of the light towards a light sensitive sensor; wherein the first surface and the second surface are provided at least partly inclined relative to each other; and

d) detecting the light generated by the scintillator elements by a light-sensitive sensor arrangement.

According to an aspect, a light guide is provided with an inclined surface to allow focusing of a scintillator. Hence, the scintillator can be arranged in discrete portions that are each focused on an X-ray source. The scintillator can be manufactured with a perpendicular grid structure, as well as the light guide that can also be provided with a perpendicular grid structure.

According to an aspect, in dark-field X-ray imaging, phase-contrast and dark-field imaging of the lung during a 2D radiography examination can be enabled. One option is to use a large size absorber grating G2, which covers the entire X-ray detector, which may be arranged in the size of 430 mm (millimeter)×430 mm. In order to absorb sufficiently radiation for dark-field imaging at clinically relevant X-ray energies, e.g. up to 125 keV, high gratings, i.e. with a high aspect ratio, are needed. Another option are so-called structured scintillators, where the scintillator is actually a grating that converts incident X-rays only at the desired locations in the first place. These structured scintillators are provided in relation with a light guide between the structured scintillator and a light sensor, such as a photo diode array, e.g. a CMOS sensor. The light guide may be provided as a fiber optic plate. As a side effect, in a fiber optic plate it is possible to absorb X-rays which are not absorbed in the scintillator and thus to avoid undesired direct conversion in the photo diode array, which would create additional noise and radiation damage. In an example, structured scintillators can be manufactured in two main steps: In the first step, a grating negative is produced using dry etching into a Si wafer. In a second step, the trenches of the grating are filled with CsI. Even though the etching process provides gratings with grating walls perpendicular to the wafer surface, the inclined optical guide allows to focus the structured scintillators to the X-ray source. The optical guide can be provided as wedge-shaped fiber optical plates. In an example, structured scintillators are provided as Si wafers with a size of 80 to 100 mm. Therefore, in an example, the detector comprises 5×5 or 4×4 tiles.

These and other aspects of the present invention will become apparent from and be elucidated with reference to the embodiments described hereinafter.

### BRIEF DESCRIPTION OF THE DRAWINGS

Exemplary embodiments of the invention will be described in the following with reference to the following drawings:

FIG. 1 shows a cross section of an example of a light guiding plate with an inclined surface.

FIG. 2 shows a cross section of a further example of a light guiding plate with a concave surface that can be focused on a common focal point.

FIG. 3 shows a cross section of an example of a light guiding plate arranged in plane segments.

FIG. 4 shows a cross section of an example of an X-ray detector with a structured scintillator, an optical guide and a sensor, wherein the optical guide is providing an inclination for the structured scintillator.

FIG. 5 shows a cross section of a detail of a structured scintillator.

FIG. 6 shows a cross section of another example of an X-ray detector.

FIG. 7 shows a schematic setup of an example of an X-ray imaging system.

FIG. 8 shows some geometrical relations in the content of lung examination.

FIG. 9 shows an example of a method for detecting X-ray radiation for medical X-ray imaging.

### DETAILED DESCRIPTION OF EMBODIMENTS

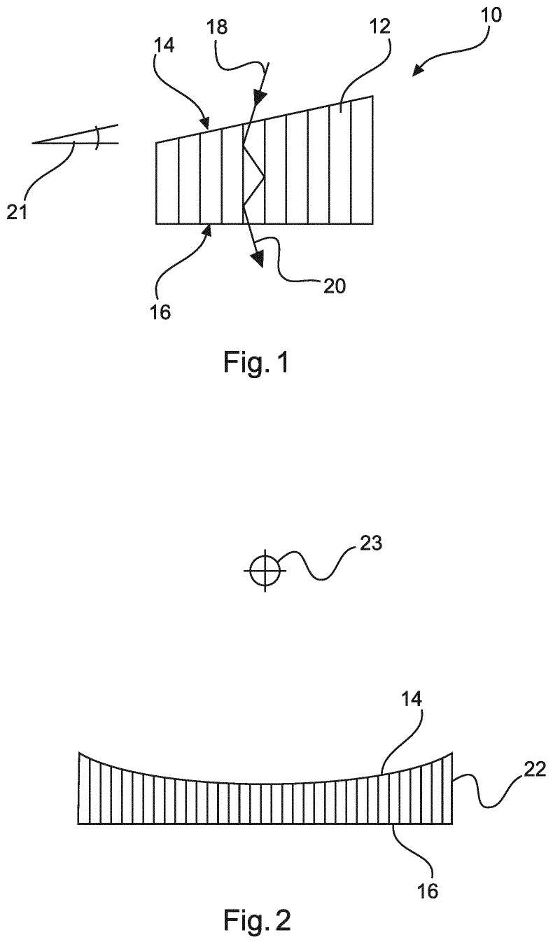

FIG. 1 shows a cross section of an example of a light guiding plate 10 for an X-ray detector. The light guiding plate 10 comprises a plurality of light guide elements 12 arranged extending between a first surface 14 of the light guiding plate 10 and an opposite second surface 16 of the light guiding plate 10. The first surface 14 is configured for entry of light generated by a scintillator to be arranged on top of the light guiding plate. The entry of the light is indicated with a first arrow 18. The second surface 16 is configured for exit of the light towards a light sensitive sensor to be arranged adjacent the light guiding plate 10. The exit of the light is indicated with a second arrow 20. The light guides elements 12 are configured to guide light from the first surface 14 layer to the second surface 16. The first surface 14 and the second surface 16 are provided at least partly inclined relative to each other. The inclination is indicated with an angle 21.

The first surface of the light guiding plate is also referred to as the first plate-surface. The second surface of the light guiding plate is also referred to as the second plate-surface.

In an example, the light guiding plate is provided as a dark-field X-ray light guiding plate for a dark-field X-ray scintillator that is arranged as a grid-like structure.

In an example, indicated in FIG. 2, the second surface 16 is arranged as a plane surface and the first surface 14 is configured as a concave surface.

In an example, shown in FIG. 2, the light guiding plate 10 is provided as a continuous plate 22.

As an option, the first surface 14 is arranged to be aligned to a focus 23.

In an example, the concave surface is formed as a part-cylindrical surface, or as a part of a cylinder. The concavity is thus provided only in one direction. The first surface is thus focussed on a linear focal line.

For example, the first surface and the second surface have a centre portion that is arranged parallel to the second surface, wherein in circumferential or outer spaces, the first surface is provided concave in one direction.

In an example, the concave surface is arranged to focus on a common focal point in which an X-ray tube can be located.

In an example, the second surface is arranged as a contoured profile to match with a contour of a sensor surface.

The light guide elements are also referred to as light-guides.

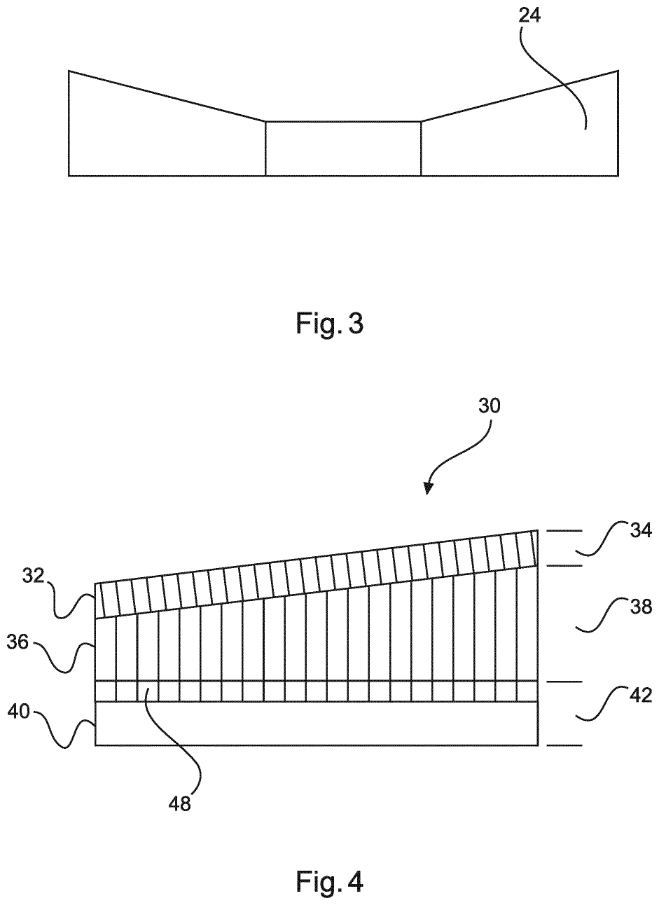

FIG. 3 shows an example of the light guiding plate comprising a plurality of plate segments 24, which are provided with different inclination of their first surface in relation to their second surface. In a basic version, three plate segments 24 are provided. In another, more complex version, more than three, such as four, five, six, seven, eight, nine, or ten ore more segments are provided in one direction. In an option, one to three or more than three, such as four, five, six, seven, eight, nine or ten ore more segments are provided in the other direction.

In an example, a plurality of plate segments is provided, which are wedge-shaped to provide a focussing on a common focal area; perpendiculars or the first surfaces are focussed to the common area.

In an example, the light guide elements are provided perpendicular to the first surface.

In an example, the light guide elements are provided perpendicular to the second surface.

In an example, the light guide elements are provided as linear tube-like elements.

In an example, the light guide elements are provided parallel to each other.

In an example, the light guide elements are provided inclined to each other such that they are focussed on a common region or common focal point in which an X-ray source can be arranged.

The terms “first” and “second” in relation to sides and surfaces usually relate to a main radiation direction.

In an example, not further shown, the light guide elements are provided as optical fibres configured to guide and transmit light between their ends. The optical fibers provide total reflection along their outer boundaries for light rays guided from the first to the second surface within the fibre.

In an example, the optical fibres extend from the first 14 to the second surface 16. The light guiding plate is also referred to as fibre-optical-plate.

In another example, also not further shown, the light guiding plate comprises a light transparent base material arranged within a grid-like pattern of wall segments that provide total reflection for light rays guided from the first to the second surface.

In an example, the light transparent base material extends from the first 14 to the second surface 16. The light guiding plate is also referred to as optical-plate.

In an example, the base material extends from the first to the second surface. The transparent base material between the wall segments thus provide the light guide elements.

FIG. 4 shows an example of an X-ray detector 30. The X-ray detector 30 comprises a scintillator arrangement 32 in a first layer 34; and an optical guide arrangement 36 in a second layer 38; and a light-sensitive sensor arrangement 40 in a third layer 42. The second layer is provided between the first and the third layer.

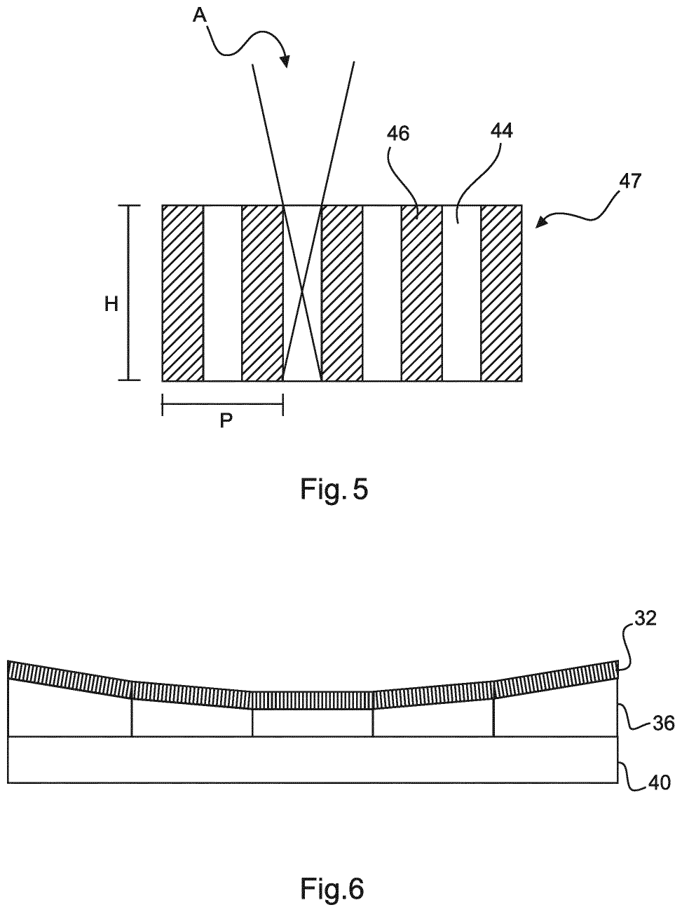

As also shown in FIG. 5, The scintillator arrangement comprises a structured scintillator with a plurality of scintillator elements 44 arranged as trenches separated from each other by wall elements 46 in a grating structure 47. The trenches are configured to receive X-ray radiation from one side and to emit visible light on a second side. As an example, the grating structure 47 has a period P of 30 μm (micrometer) and a height H of 300 μm. The grating structure 47 is provided with an opening angle A of:

30 μm/300 μm=100 mrad (milliradians)=5.7°

Referring back to FIG. 4, the light-sensitive sensor arrangement comprises a plurality of sensor elements 48 that detect light generated by the scintillator elements. In a preferred example, the sensor elements are larger than the fibers shown in the figure. The optical guide arrangement comprises a light guiding plate according to one of the examples mentioned above.

The trenches can be configured as trenches with a bottom surface. The trenches can also be configured as grooves or gaps in the grid without a bottom surface provided by a structural material. The base material can be differently shaped, as long as the resulting scintillator elements are bar-like elements separated from each other in terms of light generation.

In an example, the grating structure is provided as a grid-like structure with bars between which grooves are formed that are filed with scintillator material, i.e. material that generates visible light upon radiation with X-rays, for example CsI.

The grating structure may be provided as a Si grating template.

Light generated by the scintillator elements is guided towards the sensor elements.

In one example, the number of the scintillator elements is provided in a manner aligned with the number of the light guiding elements of the optical guide arrangement.

The scintillator arrangement in the first layer is arranged abutting the optical guide arrangement in the second layer; and the optical guide arrangement in the second layer is arranged abutting the light-sensitive sensor arrangement in the third layer.

A first transition layer is thus provided between the first layer and the second layer, and a second transition layer is thus provided between the second layer and the third layer.

The side of the scintillator arrangement in the first layer opposite to the side where the first layer is abutting the optical guide arrangement in the second layer, i.e. the side opposite to the first transition layer, is configured as an X-ray radiation entry or impinging side, which is provided to be facing an X-ray source. The side of the scintillator arrangement in the first layer where the first layer is abutting the optical guide arrangement in the second layer, i.e. the side opposite to the X-ray radiation entry side, is configured as a primary light emitting side.

The side of the optical guide arrangement in the second layer abutting the scintillator arrangement in the first layer is configured as a light entry side. The side of the optical guide arrangement in the second layer abutting the light-sensitive sensor arrangement in the third layer, i.e. the side opposite the light entry side, is configured as a light exit side or light emitting side.

The side of the light-sensitive sensor arrangement in the third layer abutting the optical guide arrangement in the second layer is configured as a light entry side.

By arranging the scintillator on the inclined light guiding plate, a scintillator grid can be arranged focussed on an X-ray source.

In an example, a detector area of 430 millimetres x 430 millimetres is provided. For example, the X-ray source provides a fan-shaped beam with a fan-angle of around 100 millirad.

FIG. 6 shows an example of the X-ray detector 30 with the scintillator arrangement 32, the optical guide arrangement 36 and the light-sensitive sensor arrangement 40. The scintillator arrangement 32 comprises a plurality of segments. The optical guide arrangement 36 also comprises a plurality of segments. As an option, the number of the segments is matching. In another option, the number of the segments of the scintillator arrangement 32 is n-fold higher, such as two segments of the scintillator arranged on one segment of the optical guide arrangement 36.

The light-sensitive sensor arrangement 40, i.e. the sensor, may be arranged as one sensor or as a plurality of sensor segments.

In an option, the number of the individual scintillator elements is matching with the number of optical guide elements.

In another option, the number of the individual scintillator elements is an n-fold of the number of optical guide elements.

In a further option, the number of the individual optical guide elements is an n-fold of the number of the scintillator elements.

In a still further option, the numbers of the elements are not matching and are not n-folds by even numbers.

As the scintillator is arranged as a grid or at least grid-like structure concerning the optical properties, an alignment fulfilling higher demands is provided.

In an example, the trenches of the scintillator are arranged focused on a common point in which an X-ray source is arrangeable. As an example, the trenches are arranged parallel to the respective adjacent light guide elements. For example, the trenches are arranged parallel to the axis that the respective segment of the light guiding plate focusses to. For example, the scintillator elements are provided as linear elements in direction from a first surface of the scintillator arrangement to a second surface of the scintillator arrangement.

The first surface of the scintillator arrangement is also referred to as the first scintillator-surface. The second surface of the scintillator arrangement is also referred to as the second scintillator-surface. In another example, the scintillator elements are arranged perpendicular to the first surface of the scintillator arrangement.

In an example, the first and second surface of the scintillator arrangement are parallel to each other and the scintillator elements are also arranged perpendicular to the second surface of the scintillator arrangement.

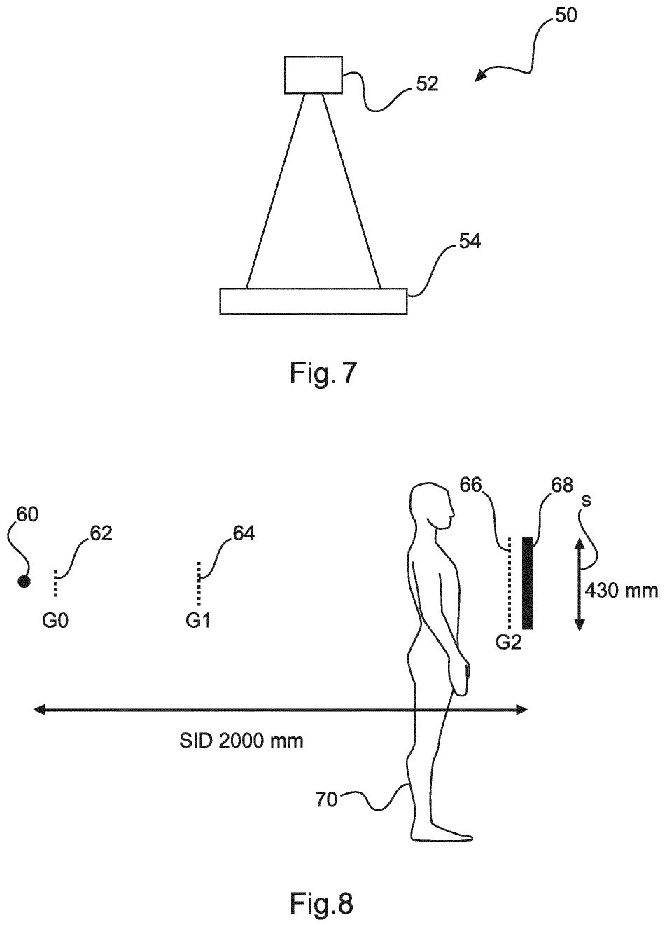

FIG. 7 shows a schematic setup of an X-ray imaging system 50. The X-ray imaging system 50 comprises an X-ray source 52 and an X-ray detector 54. The X-ray source 52 is configured to generate X-ray radiation that is detectable by the X-ray detector 54. The X-ray detector 54 is provided as an X-ray detector according to one of the examples above.

The X-ray detector is provided as a passive or indirect detector where X-ray is first converted into visible light and wherein the visible light is then detected.

In an option, a focal spot of the X-ray source is arranged in the focus of the light guiding plate.

FIG. 8 shows a geometrical setup of an X-ray imaging arrangement for lung examination. An X-ray source 60 is provided followed by a source grating 62, also referred to as G0, and a second grating 64, referred to as G1. A third grating 66, i.e. grating G2, is arranged in front of a detector plane 68. In an example, G2 is provided in form of above-mentioned structured scintillator. A patient 70 is arranged between G1 and G2. A distance SID between the source 60 and the detector plane 68 is provided to be 2000 mm. The detector can have a size S of 430 mm. A resulting system cone- and fan-angle would then be:

430:(2×2000)=108 mrad

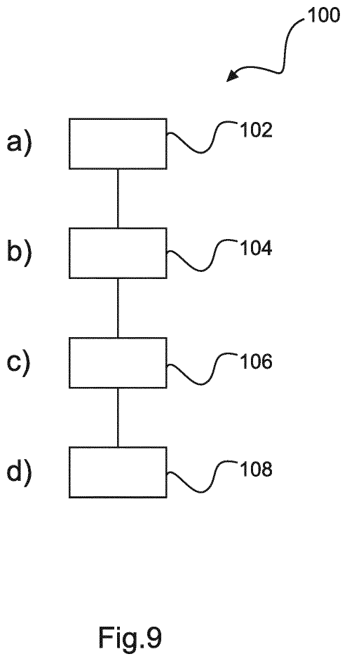

FIG. 9 shows an example of a method 100 for detecting X-ray radiation for medical X-ray imaging. The method 100 comprises the following steps:

In a first step 102, also referred to as step a), X-ray radiation is received in a scintillator arrangement in a first layer having a structured scintillator with a plurality of scintillator elements arranged as trenches separated from each other by wall elements in a grating structure. The X-ray radiation is at least partly transferred by generating visible light. In a second step 104, also referred to as step b), the visible light is emitted by the scintillator arrangement on a second side. In a third step 106, also referred to as step c), the visible light is received and guided from a first surface configured for entry of light generated by the scintillator to a second surface configured for exit of the light towards a light sensitive sensor. The first surface and the second surface are provided at least partly inclined relative to each other. In a fourth step 108, also referred to as step d), the light generated by the scintillator elements is detected by a light-sensitive sensor arrangement.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An X-ray detector, comprising:
a scintillator arrangement in a first layer, wherein the scintillator arrangement comprises a structured scintillator with a plurality of scintillator elements arranged as trenches separated from each other by wall elements in a grating structure, wherein the trenches are configured to receive X-ray radiation from one side and emit visible light on a second side, wherein the trenches are arranged to focus on a common point in which an X-ray source is arrangeable;
an optical guide arrangement in a second layer, wherein the optical guide arrangement comprises a light guiding plate which comprises a plurality of light guide elements arranged to extend between a first surface of the light guiding plate and an opposite second surface of the light guiding plate, wherein the first surface is configured for entry of light generated by the scintillator to be arranged on top of the light guiding plate, wherein the second surface is configured for exit of the light, wherein the light guides elements are configured to guide light from the first surface to the second surface, wherein the first surface and the second surface are provided at least partly inclined relative to each other; and
a light-sensitive sensor arrangement in a third layer, wherein the light-sensitive sensor arrangement comprises a plurality of sensor elements that detect light generated by the scintillator elements;
wherein the second layer is provided between the first and the third layer.

2. The X-ray detector according to claim 1, wherein the second surface is arranged as a plane surface and the first surface is configured as a concave surface.

3. The X-ray detector according to claim 2, wherein the first surface is arranged to be aligned to a focus.

4. The X-ray detector according to claim 1, wherein a plurality of plate segments is provided with different inclination of their first surface in relation to their second surface.

5. The X-ray detector according to claim 1, wherein the light guide elements are provided as optical fibers configured to guide and transmit light between their ends; wherein the optical fibers provide total reflection along their outer boundaries for light rays guided from the first to the second surface within the fiber.

6. The X-ray detector according to claim 5, wherein the optical fibers extend from the first surface to the second surface.

7. The X-ray detector according to claim 1, further comprising a light transparent base material arranged within a grid-like pattern of wall segments that provide total reflection for light rays guided from the first to the second surface.

8. The X-ray detector according to claims 1, wherein the scintillator elements are linear in direction from the first surface of the scintillator arrangement to the second surface of the scintillator arrangement.

9. The X-ray detector according to claim 1, wherein the scintillator elements are arranged perpendicular to the first surface of the scintillator arrangement.

10. An X-ray imaging system, comprising:
an X-ray source; and
an X-ray detector that comprises:
a scintillator arrangement in a first layer, wherein the scintillator arrangement comprises a structured scintillator with a plurality of scintillator elements arranged as trenches separated from each other by wall elements in a grating structure, wherein the trenches are configured to receive X-ray radiation from one side and emit visible light on a second side, wherein the trenches are arranged to focus on a common point in which an X-ray source is arrangeable;
an optical guide arrangement in a second layer, wherein the optical guide arrangement comprises a light guiding plate which comprises a plurality of light guide elements arranged to extend between a first surface of the light guiding plate and an opposite second surface of the light guiding plate, wherein the first surface is configured for entry of light generated by the scintillator to be arranged on top of the light guiding plate, wherein the second surface is configured for exit of the light, wherein the light guides elements are configured to guide light from the first surface to the second surface, wherein the first surface and the second surface are provided at least partly inclined relative to each other; and
a light-sensitive sensor arrangement in a third layer, wherein the light-sensitive sensor arrangement comprises a plurality of sensor elements that detect light generated by the scintillator elements;
wherein the second layer is provided between the first and the third layer;
wherein the X-ray source is configured to generate X-ray radiation that is detectable by the X-ray detector.

11. The X-ray imaging system according to claim 10, wherein a focal spot of the X-ray source is arranged in the focus of the light guiding plate.

12. A method for detecting X-ray radiation for medical X-ray imaging, comprising:
receiving X ray radiation on a first side in a scintillator arrangement in a first layer having a structured scintillator with a plurality of scintillator elements arranged as trenches separated from each other by wall elements in a grating structure, wherein the trenches are arranged to focus on a common point in which an X-ray source is arrangeable, wherein the X-ray radiation is at least partly transferred by generating visible light;
emitting the visible light by the scintillator arrangement on a second side;
receiving and guiding the visible light from a first surface of an optical guide arrangement configured for entry of light generated by the scintillator to a second surface configured for exit of the light towards a light sensitive sensors wherein the first surface and the second surface are provided at least partly inclined relative to each other; and
detecting the light generated by the scintillator elements by a light-sensitive sensor arrangement.
